(19) United States
(12) Patent Application Publication
(10) Pub. No.: US 2005/0267321 A1
(43) Pub. Date: Dec. 1, 2005

(54) Elastomeric magnetic nanocomposite biomedical devices

(76) Inventor: John H. Shadduck, Menlo Park, CA (US)

Correspondence Address: John H. Shadduck, 350 Sharon Park Drive #B23, Menlo Park, CA, 94025, US

(21) Appl. No.: 11/141,771

(22) Filed: Jun. 1, 2005

Related U.S. Application Data
(60) Provisional application No. 60/575,984, filed on Jun. 1, 2004.

(51) Int. Cl. A61N 2/00; A61M 37/00

(52) U.S. Cl. 600/12

(57) ABSTRACT

A biomedical device of a smart elastomer, more particularly relating to a class of low modulus elastomers with dispersed, aligned magnetic nanoparticles therein that allow for controlling the flexural modulus of the device and engaged tissue in response to an applied magnetic field. An exemplary embodiment is used for treating obstructive airway syndrome wherein one or more implants including an elastomer magnetic nanocomposite are placed in a patient's soft palate. During sleep, a source of magnetic flux is applied to stiffen the implants to dampen vibrations in tissue which occur in snoring and sleep apnea episodes. The magnetic flux is provided by a permanent magnet or by a magnetic field source coupled to a controller for modulating the stiffness of the implant(s). In similar embodiments, the controlled modulus implants can be used to treat various anatomic structures such as upper airway tissue, oral cavity tissue, gastrointestinal tract tissue, urinary tract tissue, cardiovascular tissue, muscle tissue, penile tissue, sphincters and skin.

### CROSS-REFERENCE TO RELATED APPLICATION

This application claims benefit of Provisional U.S. Patent Application Ser. No. 60/575,984 filed Jun. 1, 2004 titled Elastomeric Magnetic Nanocomposite Biomedical Devices, which is incorporated herein by reference.

### BACKGROUND OF THE INVENTION

1. Field of the Invention

This invention relates to smart elastomers for biomedical devices and more particularly relates to a class of low modulus elastomers with dispersed, magnetically aligned nanoparticles therein that can provide for controlled flexural modulus in response to an applied magnetic field. In an exemplary method of use, biomedical implants of the elastomer magnetic composite can be placed in a patient's soft palate to dampen vibrations in the tissue when sleeping in a treatment for obstructive airway syndrome, which included sleep apnea and snoring.

2. Background of the Invention

There are many clinical needs for altering the deformability of elastic tissues and body structures. In all cases, the prior art has been directed to inventions that are adapted for static tissue modifications. For example, numerous inventions relate to “tissue bulking” by means of various injectable materials and by means of creating “stiffening” lesions in tissue. Such lesions have been created by thermotherapies, cryotherpies and chemotherapies. In such so-called tissue-bulking therapies, the objective often is to alter a mechanical property of targeted tissue, such as stiffness, flexibility or more generally elasticity.

Several surgical procedures utilize such tissue-bulking methods. For example, in the field of sleep apnea and snoring, various thermal treatments and implanted materials have been proposed for creating stiffened regions in the soft palate. In other procedures, tissue-bulking has been developed for treating sphincter tissues to assist in sphincter closure. In the field of urinary incontinence treatments, both injectable materials and thermal treatments have been developed for bulking the periurethral tissues and spaces. In the gastrointestinal field, various injectables and thermal treatments are used for altering the flexibility of the lower esophageal sphincter (LES) to treat gastroesophageal reflux disease (GERD). It has been suggested that the pyloris can be bulked-up with injectables to increase the gastric retention period in a strategy to treat morbid obesity. Other bulking treatments are proposed for treating fecal incontinence by thermally-created lesions in anal sphincter tissue

In all of the prior art devices and methods, the only result of the various treatments consists of a tissue mass that has a different “static” mechanical property—for example, a stiffer, less flexible tissue. What is needed for treating many disorders that relate to tissue flexibility is a system and method for controlled, dynamic adjustment of the elastic properties of targeted tissues.

Obstructive airway syndrome sleep apnea and snoring. To understand the related art as well as to understand one embodiment of the invention disclosed herein, it is useful to refer to views of a patient's airways as shown in FIG. 1A. In normal breathing, air passes through a patient's nose or mouth and past soft flexible anatomic structures such as the base of the tongue on one side and the soft palate, uvula, and tonsils on the other. When awake, the patient's muscles around the above-described anatomic structures tighten to prevent the structures from obstructing the patient's air passageways. During sleep, the patient's muscles relax but generally the anatomic structures still do not prevent air from flowing freely into and out of the patient's lungs. Snoring occurs when anatomic structures in the throat (e.g., base of the tongue, soft palate and uvula) are lax (as when a patient's muscles over-relax) and collapse during sleep to partly obstruct the passage of air. Referring to FIG. 1A, when air passes the partly obstructed area (air flow indicated by arrows), it can be seen that the lax anatomic structures vibrate or impact each other resulting in the sounds of snoring.

The more serious obstructed airway syndrome results in sleep apnea (“apnea” meaning no breathing). Referring again to FIG. 1A, the anatomic structures may entirely block the air passageways from both the nose and mouth. During an apnea episode, the brain will cause the patient to awaken since air is not reaching the lungs. The patient typically will awaken abruptly thus causing the muscles around the lax structures in the throat to tighten and remove the obstructions to the air passageway. The patient typically emits a gasp and then breathing begins again. Such apnea episodes may continue throughout the night, resulting in fragmented nonrestful sleep. An apnea patient typically will feel tired during the day, even though he or she may not recall the waking episodes. Further, such lack of air supply to the lungs can strain the patient's lungs and heart—possibly leading to disorders such as high blood pressure, heart attack or stroke.

Non-surgical treatments for sleep apnea and severe snoring include a continuous positive air pressure (CPAP) device which has a small blower connected by a flexible hose to a mask. The blower sends a steady stream of air through the patient's nose and throat to prevent the soft structures in the throat from collapsing to obstruct the airway. Such CPAP devices have the disadvantages of being inconvenient, being necessary all night (every night) and requiring adjustment over time as the patient changes weight, etc. Other treatments for mild forms of sleep apnea include a wide range of shaped oral devices. Specially trained dental professionals cooperate with sleep disorder specialists to design devices that (i) may hold the tongue forward to prevent it from blocking the throat, (ii) may hold the entire jaw forward, or (iii) may lift the soft palate and uvula to keep such structures from blocking the throat.

Several types of surgery have been developed to prevent sleep apnea or to alleviate snoring. Most such surgeries are adapted to increase the cross-section of the airway by removing anatomic structures or tissues from around the patient's throat. The most common surgery for sleep apnea and snoring is uvulopalatopharyngoplasty (UPPP) in which the tonsils, uvula and part of the soft palate are resected from the patient's throat. Still, a UPPP is not entirely successful in treating sleep apnea since tissues further back in the throat and at the base of the tongue may still block the passage of air. More recently, laser-assisted uvulopalatoplasty (LAUP) has been developed which is considered appropriate only for snoring since the procedure does not remove all tissues that may block the airways. In a LAUP, the physician uses a laser to cut out part or all of the uvula and a portion of the soft palate. The disadvantages of UPPP and LAUP procedures are significant and include bleeding, infection, tongue numbness, voice change, food and liquid flow into the nasal passageway during swallowing, and possible failure to cure sleep apnea leading to apnea without snoring (“silent apnea”).

The above invasive surgeries do not treat a key aspect of obstructive airway disorders—the large volume of lax tissue typically found around the base of the patient's tongue. Tissue resections around the base of the tongue are not attempted because of difficulty of access as well as surgical risks mentioned above. Two types of surgeries relating to the tongue are known for treating sleep apnea. Both surgeries are very invasive and risky. In one type of surgery, the patient's jaw is detached and moved forward to make the air passageway larger beyond the base of the tongue. In a second type of surgery, the tongue attachments are severed and the tongue is re-attached in a more forward position to increase the dimension of the air passageway beyond the base of the tongue.

Improved methods for treating sleep apnea (and obstructive airway syndrome, in general) are needed. In particular, treatments that deal with lax tissues in the soft palate and around the base of the patient's tongue are needed. Preferably, the improved methods are less invasive than current surgical approaches.

### SUMMARY OF THE INVENTION

The present invention relates to implants, biomedical devices and techniques for in-situ adjustment of the flexural modulus of anatomic structures in human patients to treat various disorders.

The biomedical devices or implants of the invention include an elastomeric nanocomposite body with a flexural modulus that can be controlled by an applied magnetic field. An elastomeric magnetic nanocomposite (ENM) is fabricated by dispersing and magnetically aligning nanometric magnetic particles in a crosslinked polymeric matrix. During the crosslinking or curing of the elastomer, the nanoparticles are maintained in a selected orientation. The nanocomposite when not under the influence a magnetic flux can have a very low flexural modulus. Thereafter when in use, variable levels of magnetic flux can be applied to increase the stiffness of the monolith, as well as urge the monolith toward its memory shape. In use, controlled magnetic fields or mechanical perturbations can induce the nanoparticles within the composite to align along the lines of flux.

In one exemplary embodiment, the implant and techniques can be used to treat the symptoms of sleep apnea or OAS (obstructed airway syndrome) which includes snoring. The implants can be placed in airway tissue to dynamically stiffen relaxed tissues in the soft palate or around the base of the patient's tongue for intervals during sleep. The novel treatment is adapted to replace more invasive surgical methods, such as (i) conventional uvulopalatopharyngoplasty (UPPP), or (ii) laser-assisted uvulopalatoplasty (LAUP) both of which include resection of lax airway tissues.

The description of one exemplary embodiment in the field of OAS is not limiting, and is merely used as explanatory tool to describe a single type of implant in more detail. The invention has equally important uses in treating cardiovascular disorders, GI tract disorders, urinary tract disorders, overeating disorders, and other treatments and therapies described below.

### BRIEF DESCRIPTION OF THE DRAWINGS

The features and advantages of this invention, and the manner of attaining them, will become apparent by reference to the following description of preferred embodiments of the invention taken in conjunction with the accompanying drawings, wherein:

FIG. 1A is a sectional view of the anatomic structures of a patient's upper airway tract showing the symptoms of sleep apnea or obstructed airway syndrome, and further illustrating the elastomer magnetic nanocomposite implant corresponding to the invention.

FIG. 1B is a frontal view of the patient's soft palate illustrating exemplary locations of the elastomeric magnetic nanocomposite implants.

FIG. 2A is a perspective view of an elastomeric magnetic nanocomposite implants of a shape memory polymer in a memory shape.

FIG. 2B is a perspective view the implant of FIG. 2A in a temporary shape.

FIG. 3 is a cut-away view of a patient's stomach illustrating the implantation of at least one elastomeric magnetic composite implant in a patients lower esophageal sphincter (LES).

FIG. 4 is a cut-away view of a patient's stomach illustrating the implantation of EMN implants in a the fundus of the stomach for treating obesity.

FIG. 5 is a view of a portion of a gastrointestinal tract illustrating the implantation of at least one elastomeric magnetic nanocomposite implant in the pyloric sphincter.

### DETAILED DESCRIPTION OF THE INVENTION

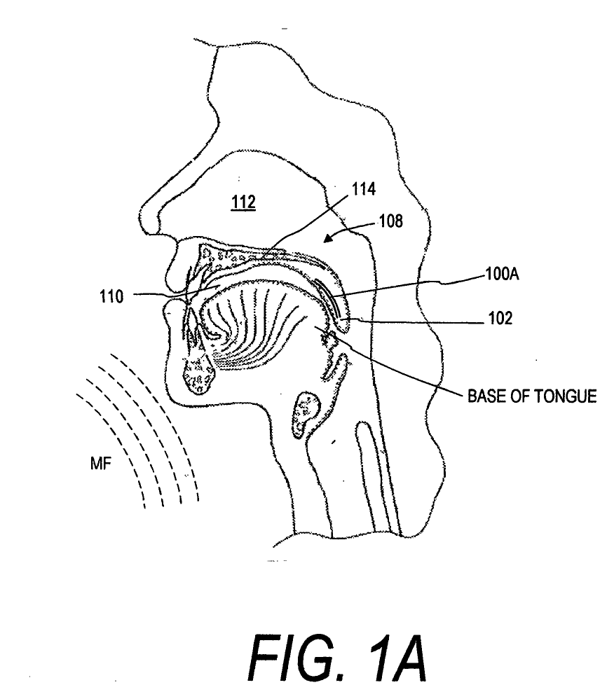

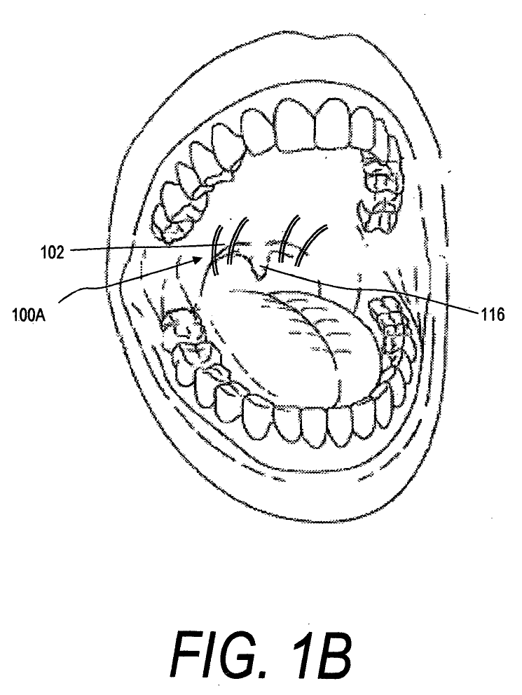

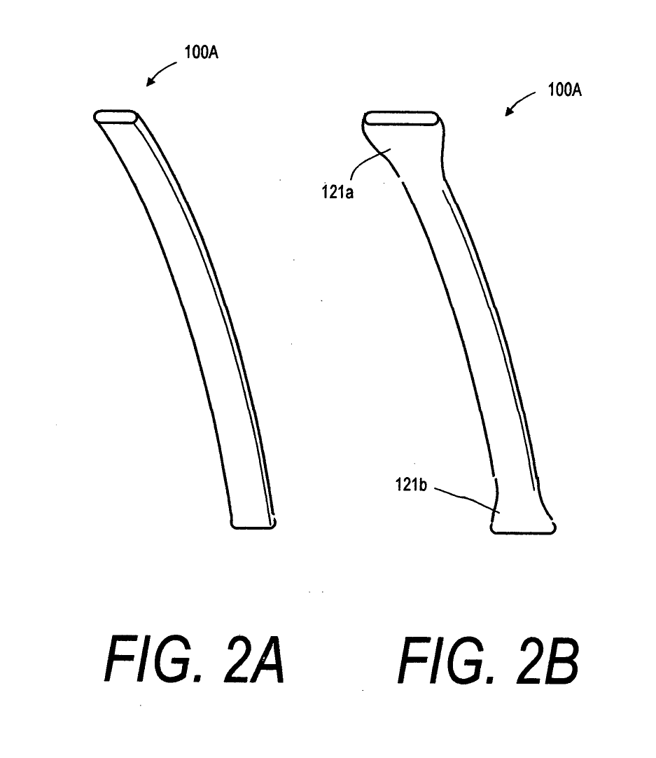

FIGS. 1A-1B and 2A-2B illustrate an exemplary embodiment of elastomer magnetic nanocomposite (EMN) and it method of use. The implant bodies 100A are shaped and formed for vibration damping in a patient's upper airway structures to treat obstructed airway syndrome (OAS) or more generally sleep apnea and snoring. The elastomer material comprises a new class of biocompatible smart material with a modulus that can be controlled by an applied magnetic field. The more detailed description of the use of an exemplary implant body 100A in treating a patient's airway tissue is not limiting, and is but one example of a number of uses in a human patient for dynamic stiffening of tissue or for controlling the flexural modulus of a target mammalian body structure.

In this disclosure, the terms “modulus” and “elastic modulus” are used to describe the flexibility and elastic properties of a composite material, and the combination of the material and engaged tissue, that can be “altered” in response to applied magnetic flux. The terms “modulus” and “elastic modulus” are use interchangeably with more specific modulus definitions such as Young's modulus and flexural modulus. In general, the system corresponding to the invention can dynamically adjust mechanical properties of a viscoelastic composition, and it is unnecessary to more specifically define the targeted modulus parameters. Young's modulus is the ratio between stress and strain, i.e., stress divided by strain wherein stress is the force per unit area acting on a material which tends to change its dimensions. Among other types, stress can be tensile as when the body is subject to a tension load, compressive as when the body is subject to compression loading, or shear as when the body is subject to a shearing load. Flexural modulus is the ratio of stress to strain within the elastic limit and is similar to the tensile modulus. Flexural modulus is used to indicate the bending stiffness of a material.

One preferred method of treating airway tissue is to insert at least one implant body 100A (and preferably from about 2 to 6 implants) of an elastomer magnetic nanocomposite (EMN) in the patient's soft palate tissue 102 as illustrated in FIGS. 1A and 1B. In FIG. 1A, the palate 108 separates the oral cavity 110 from the nasal cavity 112. The anterior region of the palate comprises the bony hard palate 114. The soft palate 102 comprises muscles and soft tissue and is suspended from the posterior portion of hard palate 114. The posterior margin of the soft palate is free to move or vibrate and terminates in the uvula 116 which droops into the posterior oral cavity and airway.

Referring to FIG. 1A, the soft palate 102 and uvula 116 are unsupported by cartilage and during sleep the muscles can relax causing these tissues to sag into the airway. The resulting airflow can cause the soft palate and uvula to vibrate which results in snoring. The soft palate and tongue also can relax sufficiently during sleep to partially or fully obstruct the airway. Such obstructive airway syndrome (OAS) can result in hypopnea wherein the airway is partially obstructed or apnea wherein the airway is completely obstructed. Sleep apnea, and to a lesser degree, hypopnea can have extremely serious health consequences.

In FIGS. 1A and 1B, the implants 100A have a modulus when free of magnetic flux that is similar to the native tissue, or is less than the native tissue, in which the implant bodies are implanted. For example, the elastic modulus can be from about 5 kPa to 5000 kPa and more preferably from about 20 kPa to 2000 kPa when not under the influence of a magnetic field or magnetic flux. In this condition, the implants would not be noticed by the patient, in terms of flexibility.

In an exemplary embodiment, the elastomer component of the nanocomposite comprises from about 30% to about 99% of the material by weight or volume, and can be any biocompatible elastomer. For example, the elastomer can comprise a cross-linked polymeric gel having the selected modulus described above and can be a thermoset or thermoplastic polymer. Suitable elastomers can comprise a silicone, a polyurethane, a hydrogel, a polyamide, a polyester, or another suitable elastomer or a combination of the above polymers. Additionally, other non-polymeric compositions can be dispersed within the nanocomposite, for biocompatibility, for prevention of tissue adherence, for antibiotic or other drug release, etc.

In an exemplary embodiment, the magnetically responsive particles or nanoparticles that are carried within the elastomer can be any suitable elements known in the art. The particle component of the composite can consist of any solid material which exhibits magnetic activity, for example any material, alloy or compounds which exhibits ferromagnetic, paramagnetic or superparamagnetic properties. Such particles or nanoparticles can be of iron, iron oxide, iron nitride, iron carbide, carbonyl iron, chromium dioxide, low carbon steel, silicon steel, nickel, cobalt, and mixtures thereof. Iron oxide includes all known pure iron oxides, such as ferric and ferrous oxides, e.g., ferrites and magnetites. The magnetic particles also comprise of alloys of iron, such as those containing aluminum, silicon, cobalt, nickel, vanadium, molybdenum, chromium, tungsten or manganese. Typically, the magnetic elements are in the form of metal powders prepared by processes well known to those skilled in the art. Many methods are available for the manufacture of metal powders, including laser pyrolysis, grinding, attrition, electrolytic deposition, metal decomposition, etc. Various metal powders are commercially available, including iron powders. In one embodiment, the particles can be iron powders, iron oxide powders or mixtures thereof and iron oxide powders and reduced iron powder mixtures. Also, reduced carbonyl iron particles are useful.

In preferred embodiments, the magnetic responsive elements are dispersed within the polymer when the polymer is in a liquid state, and the elements are then magnetically aligned with a very strong magnetic field as the elastomer polymerizes into a composite having the modulus described above. The elastomer can have any selected form or shape in which the magnetic responsive are aligned. Thus, the implant can be described as having a magnetic alignment shape under the influence of magnetic flux. In alternative embodiments, an implant body can have a uniform density or gradient in density of the magnetic particles across the implant volume. A gradient in density allows for the response of the “altered” modulus under the influence of magnetic flux to be graduated along or about an axis of the implant body.

The magnetic particle dimensions will have an influence on the response of the material to magnetic flux, and the particles have a mean cross-section ranging between 5 nm to 500 microns, and more preferably a mean cross-section ranging between about 10 nm and 100 microns. In a preferred embodiment, the magnetic responsive elements are non-spherical. Preferably, the magnetic responsive elements or nanoparticles are highly elongated. By this means, the nanoparticles will be more securely embedded in the low modulus elastomer matrix. High aspect ratio nanoparticles are fabricated by Nanogram Corporation, 2911 Zanker Road, San Jose, Calif. 95134.

While the implants are described above as treating tissue by altering the modulus or stiffness of the implant body and the engaged body structure, it is also accurate to describe the implant as being “actuated” or urged from a flexed shape toward its memory or magnetic alignment shape. Thus, the scope of the invention encompasses moving an implant body coupled to an anatomic structure toward the memory shape of an elastomer magnetic nanocomposite of the implant body.

In an exemplary embodiment, the scope of the invention encompasses an elastomeric nanocomposite wherein the composite has an elastic modulus that differs by more than 20% when under the influence of applied magnetic flux as compared to the elastic modulus when not under the influence of magnetic flux. More preferably, the nanocomposite has an elastic modulus that differs by more than 40% when under the influence of applied magnetic flux as compared to the elastic modulus when not under the influence of magnetic flux.

FIG. 1A shows a method of use the implant wherein magnetic flux MF can increase the flexural modulus of the implant 100A to stiffen the soft palate during sleep when the muscles relax. In one embodiment, the patient can wear a collar or other similar apparatus at night that carries magnetic flux means, for example, rare earth Neodymium-Iron-Boron (NIB) magnets. Such NIB magnets are very strong and one or more such magnets can be provided within a protective collar-like apparatus. During the patient's awake hours, the collar-like apparatus would not be worn and the patient would not notice the flexible filament implants. This system embodiment when worn by the patient would operate in an “always-on” mode as long as the magnetic flux is within range of the implant to thereby stiffen the soft palate.

In another embodiment, the system can use a magnetic field generator and controller for creating selected magnetic field levels and on-off intervals of magnetic flux to actuate or stiffen the implants 100A of FIGS. 1A-1B. The system can coupled to a sensor, for example, to stiffen the implants in response to the sound of snoring, or an electrode sensor that can sense electrical pattern or respiratory patterns that indicate a sleep apnea episode. The system can further control magnetic flux dosimetry in cooperation with feedback circuitry sensors again linked to electrical or respiratory signals. It should be appreciated that the application or modulation of magnetic flux can be linked to signals from any type of sensor, such an acoustic sensor, a pressure sensor, an electrical signal sensor, an accelerometer or a light sensor.

In one embodiment of airway implants depicted in FIGS. 2A and 2B, the elastomer component is of a shape memory polymer (SMP) that has a stress-free memory shape (FIG. 2B) and can formed into an internally-strained temporary shape (FIG. 2A). The implant can be a ribbon or filament having any cross-sectional shape for needle injection or insertion in the soft palate or the base of the tongue. At least one end of the implant 121a or 121b can have an increased cross-section memory shape for serving as a soft “barb” to prevent its migration after implantation. The implant still can be explanted easily. The implant also can be surface modified to prevent tissue ingrowth to allow for its extraction. In another strategy, the surface can textured for preventing migration, or have a slightly porous surface to enhance tissue ingrowth to prevent migration. The implants can be singular or plural and in any suitable shape, for example in the form of ribbons, filaments, flexible rods, discs and the like.

As background, the class of shape memory polymers (SMPs) of interest herein comprises a type of co-polymer that consists of a hard segment and a soft segment each having a different glass transition temperature. One segment has a glass transition temperature ranging between about 35° C. and 80° C. at which the shape memory polymer changes from a first dimension or volume to a second dimension or volume. For example, after implantation in tissue one segment of the polymer can have a glass transition temperature of about 35° C. to 37° so that body temperature causes the implant to self-deploy from an initial temporary shape to an expanded memory shape.

The shape memory polymers (SMPs) used in the implant body 100A (FIGS. 2A-2B) demonstrate the phenomena of shape memory based on fabricating a segregated linear block co-polymer, typically of a hard segment and a soft segment. The shape memory polymer generally is characterized as defining phases that result from glass transition temperatures in the hard and soft segments. The hard segment of SMP typically is crystalline with a defined melting point, and the soft segment is typically amorphous, with another defined transition temperature. In some embodiments, these characteristics may be reversed together with the segment's glass transition temperatures.

In one embodiment, when the SMP material is elevated in temperature above the melting point or glass transition temperature of the hard segment, the material then can be formed into a memory shape. The selected shape is memorized by cooling the SMP below the melting point or glass transition temperature of the hard segment. When the shaped SMP is cooled below the melting point or glass transition temperature of the soft segment while the shape is deformed, the temporary shape is then fixed. The original shape is recovered by heating the material above the melting point or glass transition temperature of the soft segment but below the melting point or glass transition temperature of the hard segment. (Other methods for setting temporary and memory shapes are known which are described in the literature below). The recovery of the original memory shape is thus induced by an increase in temperature, and is termed the thermal shape memory effect of the polymer. The transition temperature can be body temperature or somewhat below 37° C. in many embodiments—or a higher selected temperature when the implant body is adapted to cooperate with magnetic responsive particles or chromophores in the polymer that cooperate with a remote energy source.

The implant body 100A of FIG. 2A can change shape to the form of FIG. 2B at body temperature. Alternatively, the implant body 100A can carry any suitable biocompatible material that cooperates with photonic energy, electrical energy or magnetic energy to elevate its temperature. Light sources, Rf sources and magnetic emitters are known and can be used to deliver energy to the implant, e.g., as disclosed in the author's U.S. patent application Ser. No. 09/473,371 filed Dec. 27, 1999 (now U.S. Pat. No. 6,306,075), incorporated herein by reference. The detail of the energy source need not be further described herein. The application of energy from any source can be used with an implant that is designed to have a transition temperature anywhere above about 37° C.—for example, in a range extending from about 37° to 80° C. The step of elevating the temperature of an implant component is typically performed immediately after implantation, but the scope of the invention includes using magnetic resonant means, for example, at a later time to expand the implant component or alter the component's ability to diffuse water, or to alter other functional parameters.

Besides utilizing the thermal shape memory effect of the polymer, the memorized physical properties of the SMP can be controlled by its change in temperature or stress, particularly in ranges of the melting point or glass transition temperature of the soft segment of the polymer, e.g., the elastic modulus, hardness, flexibility, and permeability. The scope of the invention of using SMPs in implants extends to the control of such physical properties within the implant for numerous therapeutic applications.

Examples of polymers that have been utilized in hard and soft segments of SMPs include polyethers, polyacrylates, polyamides, polysiloxanes, polyurethanes, polyether amides, polyether esters, and urethane-butadiene copolymers. See, e.g., U.S. Pat. No. 5,145,935 to Hayashi; U.S. Pat. No. 5,506,300 to Ward et al.; U.S. Pat. No. 5,665,822 to Bitler et al.; and U.S. Pat. No. 6,388,043 to Langer et al, all of which are incorporated herein by reference. SMPs are also described in the literature: Ohand Gorden, Applications of Shape Memory Polyurethanes, Proceedings of the First International Conference on Shape Memory and Superelastic Technologies, SMST International Committee, pp. 115-19 (1994); Kim, et al., Polyurethanes having shape memory effect, Polymer 37(26):5781-93 (1996); Li et al., Crystallinity and morphology of segmented polyurethanes with different soft-segment length, J. Applied Polymer 62:631-38 (1996); Takahashi et al., Structure and properties of shape-memory polyurethane block copolymers, J. Applied Polymer Science 60:1061-69 (1996); Tobushi H., et al., Thermomechanical properties of shape memory polymers of polyurethane series and their applications, J. Physique IV (Colloque Cl) 6:377-84 (1996)) (all of the cited literature incorporated herein by this reference).

Of particular interest, the use of an open structure of a shape memory polymer provides several potential advantages in implants, for example, very large shape recovery strains are achievable, e.g., a substantially large reversible reduction of the Young's Modulus in the material's rubbery state; the material's ability to undergo reversible inelastic strains of greater than 10%, and preferably greater that 20% (and up to about 200%-400%); shape recovery can be designed at a selected temperature between about 30° C. and 45° C., and injection molding is possible thus allowing complex shapes. These polymers demonstrate unique properties in terms of capacity to alter the material's water or fluid permeability, thermal expansivity, and index of refraction. However, the material's reversible inelastic strain capabilities leads to its most important property—the shape memory effect. If the polymer is strained into a new shape at a high temperature (above the glass transition temperature Tₛ) and then cooled it becomes fixed into the new temporary shape. The initial memory shape can be recovered by reheating the foam above its Tₛ. The shape memory foams are of particular interest for various implants because they provide even lower density than solid SMPs.

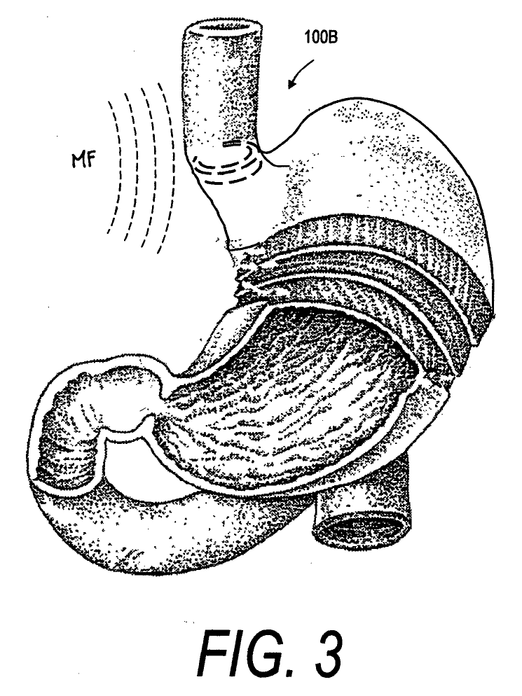

In another method and aspect of the invention, referring to FIG. 3, at least one implant body 100B can be implanted within and around the LES (lower esophageal sphincter) to allow modulation of the implant modulus and thus the flexibility, and dimensions of the sphincter. During and following food intake, or during sleep, the system can apply magnetic flux to provide an increased modulus within the implant and LES to prevent gastroesophageal reflux. The magnetic flux source can be similar to those described above.

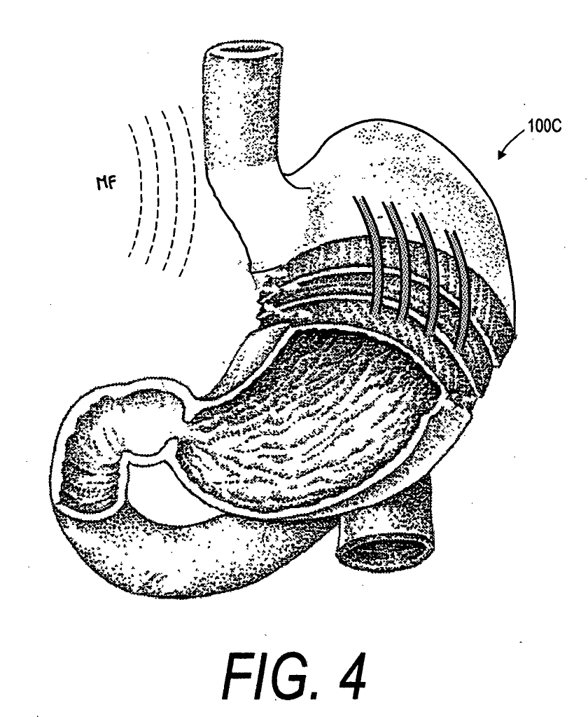

Corresponding to another method and aspect of the invention, at least one implant body 100C can be inserted into any suitable layer or layers of the stomach wall, for example interior of the mucosa or within the muscle layers as depicted in FIG. 4. The implants can be inserted with a trans-esophageal introducer, but the scope of the invention includes laparoscopic introduction from the exterior of the stomach wall. In one embodiment, the implants can be of a shape memory polymer or a non-shape memory polymer and have a flattened cross-section in a suitable elongated curved or straight shape and an elastic modulus to not substantially alter normal gastric functioning and motility. In this embodiment, a suitable magnetic flux is applied to the implants from an external source so the flexural modulus of implants is substantially increased. It is believed that such controlled stiffening of the stomach wall, together with the memory shape of the implant, can mediate the relaxation of stomach muscles in response to afferent neural signals that follow stretching of smooth muscles caused by a food bolus within the stomach. It is believed that such mediation of neurological signals will inhibit relaxation and stretching of stomach muscles to the extent that stomach volume will be inhibited during food intake. In another embodiment, magnetic responsive implants in the stomach wall can be programmed to adjust the flexural modulus of the implants episodically—which comprises a different modality than current morbid obesity treatment devices and methods that create a new static gastric condition (stomach volume reduction surgery, gastric banding, balloon deployment in the stomach, ablation of nerves, bulking of pyloris, etc.). The patient and body tissue can become accustomed to such static changes in gastric structure and function, which can result in behavioral adaptation and increased incidence of overeating.

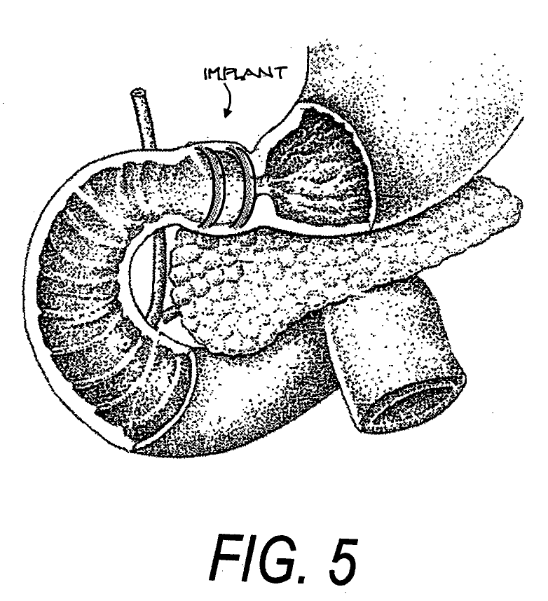

In another method and aspect of the invention, at least one implant body can be implanted within and around the pyloris to allow dynamic modulation of pyloric sphincter flexibility (see FIG. 5). During and following food intake, the system can increase the modulus of the implant and tissue region to reduce outflows to thereby increase gastric retention. The patient's feeling of satiety will thus lead to reduction in food intake.

The use of dynamically actuated implants of elastomer magnetic composites can be extended to other fields. For example, one or more implants can be implanted within and around the anal sphincter to allow modulation of the implant modulus and thus the flexibility and dimensions of the sphincter. Fecal incontinence is the second leading cause of admission to long-term care facilities in the United States—and is a devastating condition for patients. While exact data is difficult to obtain, the reported incidence rate in the general population is from 1-5%, with high rates among the elderly population. Similar system of implants can be implanted in, or coupled to, urinary tract tissue to treat incontinence. An implant of an elastomer magnetic composite corresponding to the invention can be used to control and stiffen periurethral tissue to treat stress urinary incontinence. Also, an implant of an elastomer magnetic composite can be provided in a sheet-like form to couple to and support uterine tissue in a sling or Burch procedure.

Another types of elastomer magnetic composite falls into the type of apparatus that is applied or adhered to the surface of an organ or body structure for either (i) modulus stiffening and/or (ii) actuation toward a selected shape. For example, an elastomer magnetic composite can be inserted in a heart valve wherein the system is programmed to cause dynamic stiffening of leaflets during operation of the valve. In operation, a controller would modulate magnetic flux in response to electrical signals from the heart. In such an embodiment, a pacemaker would be implanted under the skin to actuate the device. The implant would alter its modulus upon actuation as described above, and apply forces to move the implant toward its memory position, in other words providing “actuator” functionality.

In another embodiment, cylindrical members of an elastomer magnetic composite can function as penile implants to treat erectile dysfunction. The members can be implanted into the corpora cavernosa in a minimally invasive procedure and magnetic flux can be applied to stiffen the members and the engaged tissue to provide a selected modulus and shape.

Other types of stiffeners or actuator are possible, particularly for coupling temporarily or semi-permanently to skin. In facial treatments, a facial mask including an elastomer magnetic composite can be adhered gently to a patient for periodic actuation to stimulate the skin and prevent skin wrinkling. Another embodiment can be used to apply over the nostrils at night to function dynamically as a type of breath-right strip for treating snoring, and can respond to sound. Another embodiment can comprise a tubular sleeve or stent of an elastomer magnetic composite that is urged toward a non-collapsed position when under the influence of magnetic flux. Such an EMN sleeve can be inserted in any body lumen, such an airway, blood vessel, eustachian tube or the like to prop open the lumen when under the influence of magnetic flux.

The above description of the invention intended to be illustrative and not exhaustive. Those skilled in the art will appreciate that the exemplary systems, combinations and descriptions are merely illustrative of the invention as a whole, and that variations in the dimensions and compositions of invention fall within the spirit and scope of the invention. Particular features that are presented in dependent claims can be combined and fall within the scope of the invention. The invention also encompasses embodiments as if dependent claims were alternatively written in a multiple dependent claim format with reference to other independent claims. Specific characteristics and features of the invention and its method are described in relation to some figures and not in others, and this is for convenience only. While the principles of the invention have been made clear in the exemplary descriptions and combinations, it will be obvious to those skilled in the art that modifications may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

1. A therapeutic method for controlling a property of mammalian body structure comprising implanting an elastomeric magnetic composite in targeted body structure and applying magnetic flux thereby altering a property of the composite and the targeted body structure.

2. The therapeutic method of claim 1 wherein applying magnetic flux alters at least one property selected from the group of stiffness, elasticity, resilience and elastic modulus.

3. The therapeutic method of claim 1 wherein the targeted body structure is within tissue in at least one of the upper airways, the oral cavity, the gastrointestinal tract, the urinary tract, cardiovascular tissue, muscle tissue, sphincters, penile tissue or skin.

4. The therapeutic method of claim 1 wherein applying magnetic flux damps vibrations in the body structure.

5. The therapeutic method of claim 1 wherein applying magnetic flux supports body structure to thereby prevent laxity in the body structure.

6. The therapeutic method of claim 1 wherein applying magnetic flux is accomplished by means of a permanent magnet or a magnetic flux generator.

7. The therapeutic method of claim 1 wherein applying magnetic flux includes controlling parameters selected from the group of field strength and duration with a controller.

8. The therapeutic method of claim 1 wherein applying magnetic flux includes modulating the application of magnetic flux in response to a signal from at least one of an acoustic sensor, a pressure sensor, an electrical signal sensor, an accelerometer and a light sensor.

9. A biomedical device comprising a body configured for coupling to mammalian anatomic structure, the body including an elastomeric composite having an elastic modulus that differs by more than 20% when under the influence of applied magnetic flux as compared to the elastic modulus when not under the influence of magnetic flux.

10. The biomedical device of claim 9 wherein the elastic modulus differs by more than 40% when under the influence of applied magnetic flux as compared to the elastic modulus when not under the influence of magnetic flux.

11. The biomedical device of claim 9 wherein the elastic modulus ranges between about 5 kPa and 5 MPa when not under the influence of magnetic flux.

12. The biomedical device of claim 9 wherein the body is configured for coupling to tissue selected from the group of upper airway tissue, oral cavity tissue, the gastrointestinal tract tissue, urinary tract tissue, cardiovascular tissue, muscle tissue, sphincter tissue, penile tissue or skin.

13. The biomedical device of claim 9 wherein the elastomeric composite has an elastic modulus ranging between 5 kPa and 5 MPa when not under the influence of the magnetic flux.

14. The biomedical device of claim 9 wherein the elastomeric composite includes at least one of ferromagnetic, paramagnetic or superparamagnetic elements magnetically aligned in an elastomer.

15. The biomedical device of claim 14 wherein the elements have a mean cross-section ranging between 10 nm and 100 microns.

16. The biomedical device of claim 14 wherein the elastomer is at least one of a silicone, a polyurethane, a polyolefin, a polybutadiene, a natural rubber or a hydrogel.

17. A biomedical system comprising a biocompatible implant body configured for implantation in mammalian tissue, the body including an elastomer magnetic composite and a source of magnetic flux.

18. The biomedical system of claim 17 wherein the implant body has a selected shape in which the elastomer magnetic composite includes magnetically aligned elements carried therein.

19. The biomedical system of claim 17 wherein the implant body includes a shape memory polymer.

20. A biomedical system as in claim 17 wherein the source of magnetic flux is a magnet or a magnetic flux generator.
